(19) United States
(12) Patent Application Publication
(10) Pub. No.: US 2010/0322876 A1
(43) Pub. Date: Dec. 23, 2010

(54) COMPOSITIONS AND METHODS FOR TREATING KERATINOUS SUBSTRATES

(75) Inventors: Nghi Van Nguyen, Edison, NJ (US); David W. Cannell, Plainfield, NJ (US); Sawa Hashimoto, Westfield, NJ (US)

Correspondence Address: CONNOLLY BOVE LODGE & HUTZ LLP, 1875 EYE STREET, N.W., SUITE 1100, WASHINGTON, DC, 20006, US

(73) Assignee: L'Oreal, Paris (FR)

(21) Appl. No.: 12/677,246

(22) PCT Filed: Sep. 10, 2008
(86) PCT No.: PCT/US08/75851

§ 371 Date: Jul. 10, 2010

Related U.S. Application Data
(60) Provisional application No. 60/972,649, filed on Sep. 14, 2007.

(51) Int. Cl. A61Q 17/04; A61K 8/18; A61Q 15/00; A61K 8/72; A61K 8/73; A61Q 5/00

(52) U.S. Cl. 424/59; 424/62; 424/65; 424/70.9; 424/70.11; 424/70.122; 424/70.13; 424/70.14; 424/70.17

(57) ABSTRACT

The disclosure relates to compositions and methods of using the compositions to treat keratinous substrates. The compositions provide a water resistant and non-transferable protective barrier on the substrate. The compositions contain at least one polyamine, at least one acid, at least one water-insoluble ingredient, solvent and at least one auxiliary ingredient. The methods for treating keratinous substrates involve contacting the keratinous substrates with the compositions of the disclosure.

### TECHNICAL FIELD

The disclosure relates to compositions and methods for treating keratinous substrates. The compositions and methods provide a water resistant and non-transferable protective barrier on keratinous substrates imparting the substrates with improved properties.

### BACKGROUND OF THE DISCLOSURE

When keratinous substrates such as lips and skin are exposed to environmental conditions, these substrates can lose many of their desirable properties. The lips and skin can lose their moisture and become unattractive. For example, skin can lose moisture and become cracked. One method of maintaining these desirable properties is to provide a protective barrier on keratinous substrates like skin and lips. A protective moisture barrier on the skin and lips will help to keep moisture in the skin and lips allowing them to remain supple. The protective barrier is useful in cosmetic applications such as makeup, skin care and sun care products. Such a protective barrier should not be easily transferred from the substrate over time by normal everyday activity. In addition, the protective barrier should be water-resistant so that the barrier is not easily removed. Non-transfer and water-resistant cosmetic and skin care compositions are sought which have the advantage of forming a deposit which does not undergo at least partial deposition on the substrates with which they are brought into contact (for example, clothing). It is also desirable to have skin care, sun care and lip care compositions that do not easily “run off” or wash off the skin and lips when exposed to water, rain or tears. Accordingly, a product that provides a protective barrier to the substrate that also is water resistant and non-transferable would be of benefit to the area of cosmetic products such as skin care, sun care and lip care products.

### BRIEF SUMMARY OF THE DISCLOSURE

The disclosure relates to compositions for treating keratinous substrates and methods of using the compositions to treat the keratinous substrates. The disclosed compositions provide a water resistant and non-transferable protective barrier on the keratinous substrates imparting desirable properties to the substrate. The methods involve applying the compositions to the substrates. The compositions contain at least one polyamine (a), at least one acid (b), at least one water-insoluble ingredient (c), solvent (d) and at least one auxiliary ingredient (e). The methods for imparting desirable properties to the keratinous involve contacting the substrate with the compositions of the disclosure. Methods for improving the properties of keratinous substrates are also disclosed. Methods of protecting skin from UV light damage and chemical damage, keeping the skin moisturized, reducing the appearance of wrinkles and prolonging the efficacy of an active ingredient on a keratinous substrate are disclosed.

### DETAILED DESCRIPTION OF THE DISCLOSURE

The term “comprising” (and its grammatical variations) as used herein is used in the inclusive sense of “having” or “including” and not in the exclusive sense of “consisting only of”. The terms “a” and “the” as used herein are understood to encompass the plural as well as the singular.

Other than in the operating examples, or where otherwise indicated, all numbers expressing quantities of ingredients and/or reaction conditions are to be understood as being modified in all instances by the term “about”.

The term “water-insoluble” means those compounds which are either completely or partially insoluble in water.

“At least one” as used herein means one or more and thus includes individual components as well as mixtures/combinations.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The at least one polyamine of the disclosure comprises at least two amino groups and typically comprises at least five amino groups and more typically comprises at least ten amino groups.

Amino groups include primary amino groups, secondary amino groups and tertiary amino groups and further includes amino groups which are terminal, pendant and intercalated in a skeleton of the at least one polyamine compound.

The at least one acid (b) comprises at least one acid group. The at least one acid (b) may also comprise 2 or more acid groups (a polyacid).

In an embodiment of the disclosure the composition for treating a keratinous substrate comprises:
(a) at least one polyamine,
(b) at least one acid,
(c) at least one water-insoluble ingredient and
(d) solvent, and
(e) at least one auxiliary ingredient
   wherein the ratio of the amine number of the at least one polyamine to the acid number of the at least one acid is from about 1:0.5 to about 1:30 and wherein a mixture of components (a), (b), (c), and (d) form a mixture that has a contact angle of at least about 66 degrees on glass. Typically, the ratio of the amine number to the acid number is from about 1:0.8 to about 1:20 and more typically from about 1:0.9 to about 1:15 and even more typically from about 1:1 to about 1:10.

Another embodiment of the disclosure involves treating keratinous substrates with the disclosed composition. These treatments impart desirable properties to the keratinous substrate and the treatments protect the keratinous substrate.

Another embodiment of the disclosure involves treating various keratinous substrates with the disclosed composition. The methods of treating keratinous substrates with the disclosed composition includes protecting skin from UV light damage and chemical damage, reducing the appearance of wrinkles and prolonging the efficacy of an active ingredient on a keratinous substrate.

The at least one polyamine (a) may, for example, be chosen from a polyethyleneimine, a polyvinylamine, an aminated polysaccharide, an amine substituted polyalkylene glycol, an amine substituted polyacrylate crosspolymer, an amine substituted polyacrylate, an amine substituted polymethacrylate, an aminosilicone, a protein, an amine substituted polyester, a polyamino acid, an amodimethicone, a polyalkylamine, diethylene triamine, triethylenetetramine, spermidine, spermine and mixtures thereof.

Non-limiting examples of polyethyleneimine include Lupasol® products commercially available from BASF. Suitable examples of Lupasol® polyethyleneimines include Lupasol® PS, Lupasol® PL, Lupasol® PR8515, Lupasol® G20, Lupasol® G35 as well as Lupasol® SC Polyethyleneimine Reaction Products (such as Lupasol® SC-61B, Lupasol® SC-62J, and Lupasol® SC-86X). Other non-limiting examples of polyethyleneimines which may be used in the composition according to the present invention are the Epomin® products commercially available from Aceto. Suitable examples of Epomin® polyethyleneimines include Epomin® SP-006, Epomin® SP-012, Epomin® SP-018, and Epomin® P-1000. These examples include substituted polyethyleneimines.

Non-limiting examples of polyvinylamines include Lupamines® 9095, 9030, 9010, 5095 and 1595 from BASF.

An example of an amine substituted polyalkylene glycol includes PEG-15 cocopolyamine from Cognis.

An example of an aminosilicone includes Dow Corning® 2-8566 Amino Fluid, an amino functional polydimethylsiloxane fluid from Dow Corning®.

In another embodiment, the at least one polyamine compound is chosen from proteins and protein derivatives. Non-limiting examples of suitable proteins and protein derivatives for use in the present invention include those listed at pages 1701 to 1703 of the C.T.F.A. International Cosmetic Ingredient Dictionary and Handbook, 8^{th} edition, vol. 2, (2000) (incorporated herein by reference). In one embodiment, the at least one polyamine compound is chosen from wheat protein, soy protein, oat protein, collagen, and keratin protein.

In another embodiment, the at least one polyamine compound is chosen from compounds comprising lysine, compounds comprising arginine, compounds comprising histidine, and compounds comprising hydroxylysine. Not limiting examples include chitosan, polyarginine and polylysine.

An example of an amine substituted polyacrylate crosspolymer includes Carbopol® Aqua CC polymer from Lubrizol Advanced Materials, Inc.

In the present disclosure, the at least one polyamine is used in a positive amount up to about 30% by weight, more typically a positive amount up to about 10% by weight, and most typically a positive amount up to about 5% by weight, based on the weight of the composition as a whole. In some embodiments the at least one polyamine ranges from about 0.1% to about 30% by weight based on the weight of the composition. In other embodiments the at least one polyamine ranges from about 0.1 wt % to about 10 wt %, based on the weight of the composition as a whole and in further embodiments the range is from about 0.1 wt % to about 5 wt %.

The at least one acid (b) of the composition may, for example, be chosen from a fatty carboxylic acid, a fatty ether carboxylic acid, a fatty ether phosphoric acid, a fatty phosphoric acid and mixtures thereof.

Non-limiting examples of fatty carboxylic acids includes fatty acids having from about 6 to about 40 carbon atoms corresponding formula (I)

RCOOH (I)

wherein:

R is a hydrocarbon radical containing from about 6 to about 40 carbon atoms. In addition, R is linear or branched, acyclic or cyclic, saturated or unsaturated, aliphatic or aromatic, substituted or unsubstituted. Typically, R is a linear or branched, acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group, and even more typically a C₁₂₋₁₈ alkyl group or alkenyl group or a C₆₋₁₆ alkyl phenyl group.

Suitable fatty acids having from about 6 to about 40 carbon atoms include, but are not limited to the following representatives referred to by their INCI names (INCI: nomenclature for raw materials according to the International Cosmetic Ingredient Dictionary, 10^{th} Edition, published by the Cosmetic, Toiletry and Fragrance Association Inc. (CTFA), Washington D.C., USA): Arachidic Acid, Arachidonic Acid, Beeswax Acid, Capric Acid, Caproic Acid, Caprylic Acid, Coconut Acid, Isostearic Acid, Lauric Acid, Linoleic Acid, Linolenic Acid, Myristic Acid, Oleic Acid, Olive Acid, Palmitic Acid, Rapeseed Acid, Stearic Acid, Behenic Aid, Tallow Acid, Undecanoic Acid, Undecylenic Acid, 18-Methyleicosanoic Acid, Wheat Germ Acid and mixtures thereof.

Typical fatty acids having from about 6 to about 40 carbon atoms include Linoleic Acid, Oleic Acid, Isostearic Acid, and Stearic Acid.

Non-limiting examples of fatty ether carboxylic acid includes compounds corresponding to formula (II):

RO[CH₂O]ᵤ[(CH₂)ₓCH(R′)(CH₂)_{y}(CH₂)_{z}O]ᵥ[CH₂CH₂O]_{w}CH₂COOH (II)

wherein:

R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z is ≧0;

Ether carboxylic acids corresponding to formula (II) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (II), R is linear or branched, acyclic or cyclic, saturated or unsaturated, aliphatic or aromatic, substituted or unsubstituted. Typically, R is a linear or branched, acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group, and even more typically a C₁₂₋₁₈ alkyl group or alkenyl group or a C₆₋₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable ether carboxylic acids or ether carboxylates include, but are not limited to, the following representatives referred to by their INCI names (INCI: nomenclature for raw materials according to the International Cosmetic Ingredient Dictionary, 7^{th} Edition, published by the Cosmetic, Toiletry and Fragrance Association Inc. (CTFA), Washington D.C., USA): Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C₉₋₁₁ Pareth-6 Carboxylic Acid, C₁₁₋₁₅ Pareth-7 Carboxylic Acid, C₁₂₋₁₃ Pareth-5 Carboxylic Acid, C₁₂₋₁₃ Pareth-8 Carboxylic Acid, C₁₂₋₁₃ Pareth-12 Carboxylic Acid, C₁₂₋₁₅ Pareth-7 Carboxylic Acid, C₁₂₋₁₅ Pareth-8 Carboxylic Acid, C₁₄₋₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof.

Typical Carboxylic Acids are Oleth-10 Carboxylic Acid, Laureth-5 Carboxylic Acid and Laureth-11 Carboxylic Acid.

Non-limiting examples of fatty phosphoric acids include compounds corresponding to Formula III:

R—O—P(O)(OH)₂ (III)

wherein:

R is a hydrocarbon radical containing from about 6 to about 40 carbon atoms. In addition, R is linear or branched, acyclic or cyclic, saturated or unsaturated, aliphatic or aromatic, substituted or unsubstituted. Typically, R is a linear or branched acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group and most typically a C₁₂₋₁₈ alkyl group or alkenyl group or a C₆₋₁₆ alkyl phenyl group.

Typical fatty phosphoric acids include capryl phosphate, caprylyl phosphate, lauryl phosphate, oleyl phosphate, isostearyl phosphate, stearyl phosphate and cetyl phosphate.

Non-limiting examples of fatty ether phosphoric acids compounds corresponding to formulas IV and V:

RO[CH₂O]ᵤ[(CH₂)ₓCH(R′)(CH₂)_{y}(CH₂)_{z}O]ᵥ[CH₂CH₂O]_{w}—PO—(OH)₂ Formula IV,

{RO[CH₂O]ᵤ[(CH₂)ₓCH(R′)(CH₂)_{y}(CH₂)_{z}O]ᵥ[CH₂CH₂O]_{w}}₂PO—(OH) Formula V

and combinations thereof,

wherein:

R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z being ≧0.

The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formulas IV and V, R is linear of branched, acyclic or cyclic, saturated or unsaturated, aliphatic or aromatic, substituted or unsubstituted, typically a linear or branched, acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group, even more typically a C₁₂₋₁₈ alkyl group or alkenyl group or a C₆₋₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Typical fatty ether phosphoric acids include PPG-5-Ceteth-10 phosphate (CRODAFOS SG), Oleth-3 phosphate (CRODAFOS N3 acid), Oleth-10 phosphate (CRODAFOS N10 acid), and a mixture of Ceteth-10 phosphate and Dicetyl phosphate (CRODAFOS CES) all sold by Croda.

Examples of the at least one acid (b) that contain 2 or more acid groups include Acrylates Copolymer, Acrylates/Octylacrylamide Copolymer, Acrylates/Octylacrylamide/Diphenyl Amodimethicone Copolymer, Octylacrylamide/acrylates/Butylaminoethyl Methacrylate Copolymer, Acrylates/Stearyl Acrylate/Ethylamine Oxide Methacrylate Copolymer, Acrylates/t-Butylacrylamide Copolymer, PEG/PPG-25/25 Dimethicone/Acrylates Copolymer, VA/Butyl Maleate/Isobornyl Acrylate Copolymer, PVM/MA Copolymer, Ethyl ester of PVM/MA Copolymer, Butyl Ester of PVM/MA Copolymer, VA/Crotonates Copolymer, VA/Crotonates/Vinyl Neodecanoate Copolymer, VA/Vinyl Butyl Benzoate/Crotonates Copolymer, Carbomer, Polystyrene sulfonic acid, Terephthalylidene Dicamphor Sulfonic Acid, Phenylbenzimidazole Sulfonic Acid, Polyacrylamidomethylpropane Sulfonic Acid, Dimethicone PEG-7 Phosphate, Dimethicone PEG-8 Phosphate, Dimethicone PEG-10 Phosphate, Dimethicone PEG/PPG-7/4 Phosphate, Dimethicone PEG/PPG-12/4 Phosphate, Lauryl dimethicone PEG-10 Phosphate, Polyperfluoroethoxymethoxy Difluoroethyl PEG Phosphate, Polyperfluoroethoxymethoxy PEG-2 Phosphate, Polyphosphorylcholine Glycol Acrylate, Cocoamphodipropionic Acid, Lauroamphodipropionic Acid, Lauriminodipropionic Acid, Polyacrylic Acid, Polymethacrylic Acid, Polyglutamic acid, Myristoyl Glutamic Acid, Lauroyl Glutamic Acid, Palmitoyl Glutamic Acid, Cocoyl Glutamic Acid.

The at least one acid (b) is present in the composition in a positive amount up to about 50% by weight, typically a positive amount up to about 30% by weight, and more typically a positive amount up to about 15% by weight, based on the weight of the composition as a whole. In other embodiments, the at least one acid (b) is present in the composition in a range of from about 2 to about 50% by weight and in a range from about 5 to about 15% by weight, based on the weight of the composition as a whole.

The at least one water-insoluble ingredient (c) may, for example, be chosen from an oil, a polymer, a fatty ester, a hydrocarbon, a silicone, a wax, a fatty acid (in addition to the fatty acid (a)), salts of fatty acids, a fatty alcohol and mixtures thereof.

Non-limiting examples of oils include plant oil such as olive oil, avocado oil, coconut oil, aloe vera oil, almond oil, castor oil, jojoba oil, peanut oil, sesame oil, hazelnut oil, sunflower oil, colza oil, grapeseed oil, linseed oil and palm oil.

Non-limiting examples of hydrocarbon oils include mineral oil, petrolatum, paraffins, iso-paraffins, aromatic hydrocarbons and C₁₀₋₄₀ hydrocarbons which may be aliphatic, aromatic, arylaliphatic or mixtures thereof and the aliphatic hydrocarbons may be straight chain, branched, cyclic or combinations thereof.

Non-limiting examples of silicones include phenyltrimethicone, dimethicone, cyclomethicone, dimethicone copolyol, aminosilicone, laurylmethicone copolyol, cetyl dimethicone, cetyl triethylammonium dimethicone copolyol phthalate, dimethicone copolyol lactate, silicone quaternium-13, stearalkonium dimethicone copolyol phthalate, stearaminopropyl dimethicone and polyorganosiloxanes such as polydimethylsiloxane.

Non-limiting examples of waxes include paraffin wax, beeswax, candelilla wax, carnauba wax, jasmine wax, jojoba wax and mimosa wax.

Non-limiting examples of fatty acids are the same as those described above for the at least one fatty acid described above. This includes carboxylate salts of the fatty acids listed above. The sodium, potassium, ammonium, calcium and magnesium carboxylates of the fatty acids listed above are typical examples of the carboxylate salts of the fatty acids.

Non-limiting example of fatty alcohols include compounds of formula (VI):

R—OH (VI)

where R is as described above for the at least one fatty acid.

Non-limiting fatty esters include esters formed from the fatty acid of formula (I) and C₁₋₂₂ alcohols and esters formed from the fatty alcohol of formula VI and C₁₋₂₂ carboxylic acids.

In addition, non-limiting specific examples of water-insoluble ingredients includes isopropyl palmitate, capric/caprylic triglyceride, isododecane, polyisobutylene, tocopherol, tocopherol acetate, retinol, retinyl palmitate, 2-oleamido-1,3-octadecanediol, octymethoxy cinnamate, octyl salicylate, 18-Methyleicosanoic Acid and mixtures thereof.

The at least one water-insoluble ingredient (c) is present in the composition in a positive amount up to about 50% by weight, typically a positive amount up to about 30% by weight, and more typically a positive amount up to about 15% by weight based on the weight of the composition as a whole. In other embodiments, the at least one water-insoluble ingredient (c) is present in the composition in an amount from about 0.1% to about 50% by weight and in an amount from about 0.5% to about 15% by weight based on the weight of the composition as a whole.

Solvent (d) in the composition is present in an amount from about 10% by weight to about 95% by weight, typically in an amount from about 50% by weight to about 85% by weight and more typically from about 60% by weight to 80% by weight, based on the weight of the composition as a whole. The solvent is typically water, an alcohol, a nonaqueous polar solvent, a nonaqueous nonpolar solvent or mixtures thereof.

Alcohols include ethanol, propanol and butanol. Typically, the alcohol is ethanol or isopropanol.

The compositions of the disclosure may contain from 30 to 80% by weight, typically from 40 to 60% by weight, and more typically from 40 to 50% by weight, based on the weight of the total composition, of a nonaqueous polar organic solvent. A variety of nonaqueous polar organic solvents are suitable for use in the composition. Examples are as follows.

Polyols are suitable nonaqueous polar organic solvents. For purposes of this disclosure, polyols are defined as compounds which contain three or more hydroxyl groups per molecule. Examples of suitable polyols include fructose, glucamine, glucose, glucose glutamate, glucuronic acid, glycerin, 1,2,6hexanetriol, hydroxystearyl methylglucanine, inositol, lactose, malitol, mannitol, methyl gluceth-10, methyl gluceth-20, methyl glucose dioleate, methyl glucose sesquicaprylate/sesquicaprate, methyl glucose sesquicocoate, methyl glucose sesquiisostearate, methyl glucose sesquilaurate, methyl glucose sesquistearate, phytantriol, riboflavin, sorbeth-6, sorbeth-20, sorbeth-30, sorbeth-40, sorbitol, sucrose, thioglycerin, xylitol, and mix thereof.

Also suitable as a nonaqueous polar organic solvent are homopolymeric or block copolymeric liquid ethers. Polymeric ethers are preferably formed by polymerization of monomeric alkylene oxides, generally ethylene or propylene oxides. Examples of such polymeric ethers include PEG, PPG, and derivatives thereof.

Other examples of suitable polymeric ethers include polyoxypropylene polyoxyethylene block copolymers. Such compounds are sold under the CTFA name Meroxapol 105, 108, 171, 172, 174, 178, 251, 252, 254, 255, 258, 311, 312, and 314.

Also suitable for use as to the nonaqueous polar organic solvent are mono- and dihydric alcohols of the general formula R(OH)n where n is 1 or 2 and R is a substituted or unsubstituted saturated C₂₋₁₀, preferably C₁₋₈ alkyl, or a substituted or unsubstituted alicyclic, bicyclic, or aromatic ring, with the substituents selected from halogen, alkoxy, hydroxy, and so on. Examples of suitable alcohols include monohydric alcohols such as ethanol, isopropanol, hexyldecanol, benzyl alcohol, propyl alcohol, and isopropyl alcohol, as well as dihydric alcohols such as hexylene glycol, diethylene glycol, ethylene glycol, propylene glycol, 1,2-butylene glycol, triethylene glycol, dipropylene glycol, and mixtures thereof.

Sorbitan derivatives, which are defined as ethers or esters of sorbitan, are also suitable polar solvents. Examples of suitable sorbitan derivatives are the Polysorbates, which are defined as stearate esters of sorbitol and sorbitan anhydrides, such as Polysorbate 20, 21, 40, 60, 61, 65, 80, 81, and 85. Also suitable are fatty esters of hexitol anhydrides derived from sorbitol, such as sorbitan trioleate, sorbitan tristearate, sorbitan sesquistearate, sorbitan stearate, sorbitan palmitate, sorbitan oleate, and mixtures thereof.

The compositions of the disclosure may contain from 20 to 60% by weight, preferably from 20 to 50% by weight, and more preferably from 30 to 40% by weight, based on the weight of the total composition, of a nonaqueous nonpolar organic solvent. A variety of nonaqueous nonpolar organic solvents may be used in the compositions of the disclosure.

Silicones are suitable nonpolar compounds. The silicones may be volatile or non-volatile. The term “volatile” means that the silicone has a measureable vapor pressure, i.e. a vapor pressure of at least 2 mm. of mercury at 20° C. If volatile, the silicone generally will have a viscosity of 0.5 to 25 centistokes at 25° C. Suitable volatile silicones include cyclic silicones, linear silicones, or mixtures thereof.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

The silicone may also be nonvolatile, and in particular water insoluble nonvolatile silicones. The term “nonvolatile” means that the silicone has a vapor pressure of less than 2 mm. of mercury at 20° C. A variety of silicones fit this definition including dimethicone, phenyl trimethicone, diphenyl dimethicone, methicone, hexadecyl methicone, stearoxydimethicone, stearyl dimethicone, cetyl dimethicone, and so on.

Cyclomethicone is a typical silicone for use in the compositions of the disclosure.

In addition to the sorbitan esters, other esters are also suitable as the nonaqueous nonpolar organic solvent. In general such esters have the formula RCO—OR wherein each R is independently a C1-25 straight or branched chain saturated or unsaturated alkyl, alkylcarbonyloxyalkyl, or alkoxycarbonylalkyl, aryl, which may be substituted or unsubstituted with halogen, hydroxyl, alkyl, and the like.

Examples of suitable esters include alkyl acetates, alkyl behenates, alkyl lactates, alkyl benzoates, alkyl octanoates, alkyl salicylates, and in particular C12-15 alkyl benzoate. Examples of further esters are set forth on pages 502-506 of the CTFA Cosmetic Ingredient Handbook, Second Edition, 1992, which is hereby incorporated by reference.

Fats and oils are also suitable as the nonaqueous nonpolar organic solvent. Preferably these materials are liquids or semi-solids at room temperature. They are generally defined as glyceryl esters of fatty acids (triglycerides), as well as the synthetically prepared esters of glycerin and fatty acids. Examples of such materials include oils such as apricot kernel oil, avocado oil, canola oil, olive oil, sesame oil, peanut oil, soybean oil, trilinolenin, trilinolein, trioctanoin, tristearin, triolein, sesame oil, rapeseed oil, sunflower seed oil, and so on.

Fatty acids are also suitable as the nonaqueous nonpolar organic solvent in the compositions of the invention. Preferably the fatty acids are liquid or semi solid at room temperature. Fatty acids are the carboxylic acids obtained by hydrolysis of animal or vegetable fats and oils. Carboxylic acids having alkyl chains shorter than about seven carbon atoms are not generally considered fatty acids. Fatty acids have the general structure R—COOH where R is a straight or branched chain saturated or unsaturated C₇₋₆₅ alkyl. Examples of suitable fatty acids include arachidic acid, arachidonic acid, behenic acid, capric acid, caproic acid, caprylic acid, coconut acid, corn acid, cottonseed acid, hydrogenated coconut acid, hydroxystearic acid, lauric acid, linoleic acid, linolenic acid, linseed acid, myristic acid, oleic acid, palmitic acid, palm kernel acid, soy acid, tallow acid, and the like.

Fatty alcohols may also be used as the nonaqueous nonpolar organic solvent. Fatty alcohols are generally made by reducing the fatty acid —COOH group to the hydroxyl function. They generally have the formula RCH2OH. Examples of fatty alcohols are behenyl alcohol, C₉₋₁₁ alcohol, C₁₂₋₁₃ alcohol, C₁₂₋₁₅ alcohol, C₁₂₋₁₆ alcohol, caprylic alcohol, alcohol, cetyl alcohol, coconut alcohol, decyl alcohol, lauryl alcohol, myristyl alcohol, oleyl alcohol, and the like.

Hydrocarbons are also good nonaqueous nonpolar organic solvents in accordance with the disclosure. Examples of suitable hydrocarbons include C₇₋₆₀ isoparaffins, ethane, heptane, hexane, hydrogenated polyisobutene, isobutane, isododecane, isoeicosane, isohexadecane, isopentane, microcrystalline wax, mineral oil, mineral spirits, paraffin, petrolatum, petroleum distillates, squalene, polyethylene, and mixtures thereof. Preferred hydrocarbons are mineral oil and polyethylene.

Also suitable as the nonaqueous nonpolar organic solvent are lanolin and derivatives thereof. Examples of such materials include acetylated hydrogenated lanolin, acetylated lanolin alcohol, laneth, lanolin acid, lanolin oil, lanolin alcohol and lanolin wax.

The composition contains at least one auxiliary ingredient (e) in a positive amount of up to about 50% by weight, based on the weight of the composition. The auxiliary ingredient may include proteins, amino acids, cationic conditioners, cationic polymers, nonionic surfactants, anionic surfactants, amphoteric surfactants, zwitterionic surfactants, viscosity modifiers, organosiloxane polymer, waxes, silicone resins, pigments, powders, preservatives, vitamins, antioxidants, alpha hydroxyl acids, beta hydroxyl acids, alpha keto acids, antibacterial agents, sunscreens, preservatives, pH adjusting agents, bleaching agents, perfumes, sequestering agents, anti-dandruff agents, a film forming agent, absorbants, anti-acne actives, antiperspirant actives, anti-wrinkle and antiskin atrophy actives, artificial tanning and accelerators, astringents, hydrophilic conditioning agents, hydrophobic conditioning agents, oil-soluble gelling agents, hydrophilic gelling agents, crosslinked silicone polymers and mixtures thereof.

Non-limiting examples of proteins include collagen, deoxyribonuclease, iodized corn protein, milk protein, protease, serum protein, silk, sweet almond protein, wheat germ protein, wheat protein, alpha and beta helix of keratin proteins, hair proteins, such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Non-limiting examples of amino acids include amino acids derived from the hydrolysis of various proteins as well as the salts, esters, and acyl derivatives thereof Non-limiting examples of such amino acid agents include amphoteric amino acids such as alkylamido alkylamines, i.e. stearyl acetyl glutamate, capryloyl silk amino acid, capryloyl collagen amino acids, capryloyl keratin amino acids, capryloyl pea amino acids, cocodimonium hydroxypropyl silk amino acids, corn gluten amino acids, cysteine, glutamic acid, glycine, hair keratin amino acids, amino acids such as asparatic acid, threonine, serine, glutamic acid, proline, glycine, alanine, cystine, valine, methionine, isoleucine, leucine, tyrosine, phenylalanine, cysteic acid, lysine, histidine, arginine, cysteine, tryptophan, citrulline, lysine, silk amino acids, wheat amino acids and mixtures thereof.

Non-limiting examples of cationic conditioners include quaternium-27, behenamidopropyl PG-dimonium chloride, hydroxyethyl tallowdimonium chloride, hexadimethrine chloride, stearalkonium chloride and cetrimonium chloride.

Non-limiting examples of cationic polymers include polyquaternium-4, polyquaternium-6, polyquaternium-7, polyquatemium-10, polyquaternium-11, polyquaternium-16, polyquaternium-22 and polyquatemium-32.

Non-limiting examples of nonionic surfactants includes alkoxylated derivatives of the following: fatty alcohols, alkyl phenols, fatty acids, fatty acid esters and fatty acid amides, wherein the alkyl chain is in the C₁₂₋₅₀ range, typically in the C₁₆₋₄₀ range, more typically in the C₂₄ to C₄₀ range, and having from about 1 to about 110 alkoxy groups. The alkoxy groups are selected from the group consisting of C₂₋C₆ oxides and their mixtures, with ethylene oxide, propylene oxide, and their mixtures being the typical alkoxides. The alkyl chain may be linear, branched, saturated, or unsaturated. Of these alkoxylated non-ionic surfactants, the alkoxylated alcohols are typical, and the ethoxylated alcohols and propoxylated alcohols are more typical. The alkoxylated alcohols may be used alone or in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10), steareth-10 (a stearyl alcohol ethoxylate having an average degree of ethoxylation of 10), steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Commercially available nonionic surfactants are Brij nonionic surfactants from Uniqema, Willmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, which are the condensation products of long chain alcohols, e.g. C₈-C₃₀ alcohols, with sugar or starch polymers. These compounds can be represented by the formula (S)n-O—R wherein S is a sugar moiety such as glucose, fructose, mannose, galactose, and the like; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein sucrose ester surfactants such as sucrose cocoate and sucrose laurate.

Other nonionic surfactants suitable for use in the present invention are glyceryl esters and polyglyceryl esters, including but not limited to, glyceryl monoesters, typically glyceryl monoesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Uniqema, Wilmington, Del.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use as nonionic surfactants are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being typical. Non-limiting examples of commercially available ethoxylated materials include TWEEN® (ethoxylated sorbitan mono-, di- and/or tri-esters of C₁₂ to C₁₈ fatty acids with an average degree of ethoxylation of from about 2 to 20).

Non-limiting examples of anionic surfactants include compounds in the classes known as alkyl sulfates, alkyl ether sulfates, alkyl sulfonates, alkyl ether sulfonates, sulfate esters of an alkylphenoxy polyoxyethylene ethanol, alpha-olefin sulfonates, beta alkyloxy alkene sulfonates, alkyl arylsulfonates, alkyl carbonates, alkyl ether carboxylates, fatty acids, succinamates, sulfosuccinates, sarcosinates, octoxynol or nonoxynol phosphates, taurates, fatty taurides, sulfated monoglycerides, fatty acid amino polyoxyethylene sulfates, isothienates and mixtures thereof. Specific examples of anionic surfactants include the ammonium, monoethanolamine, diethanolamine, triethanolamine, isopropylamine, sodium, potassium, lithium, or magnesium salts of lauryl sulfate, dodecylbenzene-sulfonate, lauryl sulfosuccinate, lauryl ether sulfate, lauryl ether carboxylate, lauryl sarcosinate, cocomethyl tauride, and sulfosuccinate half ester amide and mixtures thereof.

Non-limiting examples of amphoteric and zwitterionic surfactants include alkyl, alkyl dimethyl, alkylamido, alkyl amide, alkylamidopropyl, or alkyl dimethylammonium betaine; alkyl amidopropyl or alkyl sulfobetaine; alkyl, alkylampho, or alkylamphocarboxy glycinate; alkyl, or alkyl substituted imidazoline mono or dicarboxylate; sodium salts of alkyl mono-or dicarboxylates; alkyl beta amino acids; alkyl amidopropyl, or alkyl ether hydroxysultaine; alkyl amidopropyl dimethyl ammonia acetate; alkyl ampho mono-or diacetate; alkyl, or alkyl ampho, or alkyl imino dipropionate; alkyl amphopropionate; alkyl beta amino propionic acid; alkyl dipropionate; alkyl beta iminodipropionate; branched or n-alkyl dimethylamidopropionate; alkyl carboxylated propionate; alkyl, or methyl alkyl imidazoline; fluorinated alkyl amphoteric mixtures; and/or nonionic surfactants such as, but not limited to, alkyl, alkyl dimethyl, alkyl amidopropylamine, or bis 2-hydroxy ethyl alkyl amine oxides; alkanolamides; alkyl amides; polyoxyethylene glycol (PEG) of monoglycerides, of sorbitan esters, of branched or linear fatty alcohol ethers, of branched or linear fatty acid ethers, of thioethers; alkyl oxoalcohol PEG; PEG fatty esters; polyoxyethlyene glycol/polyoxpropylene glycol block copolymers; alkyl phenol PEG ethers; alkyl polyglucosides, or polysaccarides, polysiloxane polyethoxylene ether and mixtures thereof.

Specific examples include cocamidopropyl betaine, lauramidopropyl betaine, coco/oleamidopropyl betaine, lauryl betaine, coco betaine, oleyl betaine, cocamidopropyl hydroxysultaine, tallowamidopropyl hydroxysultaine, dihydroxyethyl tallow glycinate, disodium cocoamphodiacetate, disodium cocoamphodipropionate and mixtures thereof.

Non-limiting examples of viscosity modifiers include water swellable/soluble cationic polymers from quaternized polysaccharides such as trimethyl ammonium substituted epoxide of hydroxyethyl cellulose, diallyl dimethyl ammonium salts of hydroxyethylcellulose, deacylated chitin or chitosan, dihydroxypropyl chitosan trimonium chloride, hydroxypropltrimethyl ammonium chloride guar, locust bean, or konjac mannan gum; quaternized synthetics such as acrylamide dimethyl diallyl ammonium chloride copolymers, acrylamide/dimethyl diallyl ammonium chloride/acrylic acid terpolymer, quaternized poly (vinyl pyrrolidone/dimethyl amino ethylmethacrylate), poly (vinylpyrrolidone/methacrylamidopropyl trimethylammonium chloride), polyvinyl pyrrolidone/methylvinylimidazolinium chloride or methyl sulfate copolymer, chloroethylether/dimethylaminopropylamine/adipate or azelate terpolymer, vinylpyrrolidone/methacrylamidopropyl trimethylammonium chloride, acrylonitrile/acrylic acid/dimethylpropanediammonium acrylates sulfate terpolymer. Anionic or nonionic polysaccharide polymers such as gum tragacanth, sodium or propylene glycol alginate, kappa-, iota-, or lambda-carrageenan, guar or hydroxyl propyl guar gum, karaya gum, gum Arabic, locust bean gum, konjac mannan gum, gellan, xanthan, succinoglycan or its acidic or enzymatic hydrolysates, sodium carboxymethyl cellulose, methycellulose, hydroxylethylcellulose, hydroxypropylmethylcellulose, and hydroxypropylecellulose; and/or hydrophobically modified anionic, cationic, or nonionic polymers such as, but not limited to, alkyl and/or substituted hydroxyethylcellulose, lauryl dimethyl ammonium substituted epoxide of hydroxyethylcellulose, propoxylated cellulosic, xanthan, succinoglycan, or polygalactomannoses, alkyl methacrylates/crosslinked acrylic acid copolymer and/or acrylonitrile/acrylates block copolymer.

Examples of organosiloxane polymers useful in the disclosure are commercially available from Goldschmidt Corporation under the ABIL tradename. One typical example is cetyl dimethicone copolyol and has the tradename ABIL WE 09 or ABIL WS 08. The cetyl dimethicone copolyol may be used alone or in conjunction with other non-silicone organic emulsifiers. For example, the cetyl dimethicone copolyol may be used in an admixture with other non-silicone organic auxiliary ingredients such as emulsifiers and emollients. For example, the mixtures identified by the C.T.F.A. names cetyl dimethicone copolyol (and) polyglyceryl 4-isostearate (and) hexyl laurate, or cetyl dimethicone copolyol (and) polyglyceryl-3 oleate (and) hexyl laurate both work well. These blends contain approximately 25-50% of each ingredient, for example ABIL WE 09 contains approximately, by weight of the total ABIL composition, 25-50% cetyl dimethicone copolyol, 25-50%, polyglyceryl 4-isostearate, and 25-50% of hexyl laurate which is an emollient or oil.

Another type of organosiloxane polymer suitable for use in the compositions of the disclosure are sold by Union Carbide under the Silwet™ trademark. These compositions are represented by the following generic formulas:

(Me₃Si)_{y}-2[(OSiMe₂)ₓ/yO—PE]_{y}

wherein PE=-(EO)ₘ(PO)ₙR

R=lower alkyl or hydrogen

Me=methyl

EO is polyethyleneoxy

PO is polypropyleneoxy

m and n are each independently 1-5000

x and y are each independently 0-5000, and 8

wherein PE=—CH₂CH₂CH₂O(EO)ₘ(PO)ₙZ

Z=lower alkyl or hydrogen, and

Me, m, n, x, y, EO and PO are as described above,

with the proviso that the molecule contains a lipophilic portion and a hydrophilic portion. Again, the lipophilic portion can be supplied by a sufficient number of methyl groups on the polymer backbone.

Examples of other polymeric organosiloxane surfactants or emulsifiers include amino/polyoxyalkyleneated polydiorganosiloxanes disclosed in U.S. Pat. No. 5,147,578. Also suitable are organosiloxanes sold by Goldschmidt under the ABIL trademark including ABIL B-9806, as well as those sold by Rhone-Poulenc under the Alkasil tradename. Also, organosiloxane polymers sold by Amerchol under the Amersil tradename, including Amersil ME-358, Amersil DMC-287 and Amersil DMC-357 are suitable. Dow Corning surfactants such as Dow Corning 3225C Formulation Aid, Dow Corning 190 Surfactant, Dow Corning 193 Surfactant, Dow Corning Q2-5200, and the like are also suitable. In addition, products sold under the tradename Silwet by Union Carbide, and products sold by Troy Corporation under the Troysol tradename, those sold by Taiwan Surfactant Co. under the tradename Ablusoft, those sold by Hoechst under the tradename Arkophob, are also suitable for use in the disclosure.

The compositions of the disclosure may contain wax at a concentration about 0.1-25%, preferably 0.5-20%, more typically 1-15% by weight based on the total weight of the composition. Suitable waxes have a melting point of 35 to 120° C., and can be animal waxes, plant waxes, mineral waxes, silicone waxes, synthetic waxes, and petroleum waxes. Examples of waxes in accordance with the disclosure include bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG-6 beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, ethylene homo- or copolymers, stearoxy dimethicone, dimethicone behenate, stearyl dimethicone, and the like, as well synthetic homo- and copolymer waxes such as PVP/eicosene copolymer, PVP/hexadecene copolymer, and the like.

Silicone resins in the compositions of the disclosure may be added at a concentration in a range of about 0.001-20%, typically 0.01-15%, more typically 0.1-10% by weight based on the total weight of the composition. Examples of suitable silicone resins include siloxy silicate polymers having the following general formula:

[(RR′R″)₃SiO_{1/2}]ₓ[SiO₂]_{y}

wherein R, R′ and R″ are each independently a C₁₋₁₀ straight or branched chain alkyl or phenyl, and x and y are such that the ratio of (RR′R″)₃SiO_{1/2} units to SiO₂ units is 0.5 to 1 to 1.5 to 1.

Typically R, R′ and R″ are a C₁₋₆ alkyl, and more preferably are methyl and x and y are such that the ratio of (CH₃)₃SiO_{1/2} units to SiO₂ units is 0.75 to 1. For example, a trimethylsiloxy silicate containing 2.4 to 2.9 weight percent hydroxyl groups which is formed by the reaction of the sodium salt of silicic acid, chlorotrimethylsilane, and isopropyl alcohol may be used. The manufacture of trimethylsiloxy silicate is set forth in U.S. Pat. Nos. 2,676,182; 3,541,205; and 3,836,437, all of which are hereby incorporated by reference. Trimethylsiloxy silicate as described is available from Dow Corning Corporation under the tradename 2-0749 and 2-0747, which is a blend of about 40-60% volatile silicone and 40-60% trimethylsiloxy silicate. Dow Corning 2-0749 in particular, is a fluid containing about 50% trimethylsiloxy silicate and about 50% cyclomethicone. The fluid has a viscosity of 200-700 centipoise at 25° C., a specific gravity of 1.00 to 1.10 at 25° C., and a refractive index of 1.40-1.41.

Other silicone resins are silicone esters comprising units of the general formula RₐR^{E}_{b}SiO_{[4-(a+b)/2]} or R¹³ₓR^{E}_{y}SiO_{1/2}, wherein R and R¹³ are each independently an organic radical such as alkyl, cycloalkyl, or aryl, or, for example, methyl, ethyl, propyl, hexyl, octyl, decyl, aryl, cyclohexyl, and the like. a is an number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Typical R^{E} radicals are those wherein the ester group is formed of one or more fatty acid moieities (e.g. of about 6, often about 6 to 30 carbon atoms) and one or more aliphatic alcohol moieities (e.g. of about 10 to 30 carbon atoms). Examples of such acid moieities include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxypropane. Typically, the ester subgroup (i.e. the group containing the carboxylic ester) will be linked to the silicon atom by a divalent aliphatic chain that is at least 2 or 3 carbon atoms in length, e.g. an alkylene group or a divalent alkyl ether group. Most typically, that chain will be part of the alcohol moiety, not the acid moiety. More typically, the cross-linked silicone ester can be a liquid or solid at room temperature. The compound may have a waxy feel and a molecular weight of no more than about 100,000 daltons.

Such silicone resins having the above formula are disclosed in U.S. Pat. No. 4,725,658 and U.S. Pat. No. 5,334,737, which are hereby incorporated by reference. These ingredients are commercially available from General Electric under the tradenames SF 1318 and SF 1312, respectively.

Pigments and powder may be added as a auxiliary ingredient at a concentration of about 0.001-35%, typically 0.01-20% more typically 0.1-10%, by weight based the total weight of the composition. Typically the pigments and powders have a particle size of 0.02 to 200, microns typically 0.5 to 100, microns. The particulate matter may be colored or non-colored (for example white). Suitable powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone oil or various other agents either alone or in combination, which coat the powder surface and render the particles more lipophilic in nature.

The powder component also may comprise various organic and inorganic pigments. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof

The auxiliary ingredient may contain a mixture of both pigmented and non-pigmented powders. The percentage of pigments used in the powder component will depend on the type of cosmetic being formulated.

The auxiliary ingredient of the disclosure may contain 0.001-20%, typically 0.01-10%, more typically 0.05-8% of one or more sunscreens. A sunscreen is defined as an ingredient that absorbs at least 85 percent of the light in the UV range at wavelengths from 290 to 320 nanometers, but transmits UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorporated by reference. Examples of such sunscreen materials are p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)]aminobenzoate (ethyl dihydroxypropyl PABA), 2-ethythexyl-2-cyano-3,3-diphenylacrylate (octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), (Octyl Dimethyl PABA), 2-Phenylbenzimidazole-5-sulfonic acid (Phenylbenzimidazole Sulfonic acid), Red Petrolatum, Sulisobenzone (Benzophenone4), triethanolamine salicylate (TEA-Salicylates), benzophenones, bornelone, butyl PABA, cinnamidopropyl trimethyl ammonium chloride, disodium distryrylbiphenyl disulfonate, PABA, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate O and mixtures thereof.

The auxiliary ingredient may include about 0.0001-8%, typically 0.001-6%, more typically 0.005-5% by weight of a preservative based on the total weight of the composition. A variety of preservatives are suitable, including such as benzoic acid, benzyl alcohol, ethanol, polyvinyl alcohol, phenoxyethanol, methyl paraben, propyl paraben, benzylhemiformal, benzylparaben, 5-bromo-5-nitro-1,3-dioxane, 2-bromo-2-nitropropane-1,3-diol, butyl paraben, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference. The auxiliary ingredient may include mixtures of these preservatives.

The auxiliary ingredient of the disclosure may contain vitamins and/or coenzymes, as well as antioxidants. These may be added at a concentration of about 0.001-10%, typically 0.01-8%, more typically 0.05-5% by weight based on the total weight of the composition. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and mixtures thereof.

The auxiliary ingredient may include one or more alpha or beta hydroxy acids or alpha ketoacids. Typical ranges are 0.01-20%, more typically 0.1-15%, and even more typical 0.5-10% by weight based on the total composition. Suitable alpha hydroxy acids and alpha ketoacids are disclosed in U.S. Pat. No. 5,091,171, which is hereby incorporated by reference. The general structure of such alpha hydroxy acids may be represented by the following formula:

(Ra)(Rb)C(OH)COOH

wherein Ra and Rb are H, F, Cl, Br, alkyl, aralkyl, or aryl group of saturated, unsaturated, straight or branched chain or cyclic form having 1-10 carbon atoms, and in addition Ra or Rb may carry OH, CHO, COOH and alkoxy groups having 1 to 9 carbon atoms.

The alpha hydroxy acids may exist in the keto acid form, or the ester form. Examples of such alpha hydroxy acids include glycolic acid, malic acid, pyruvic acid, mandelic acid, lactic acid, methyllactic acid, and mixtures thereof.

Also beta hydroxy acids such as salicylic acid, and derivatives thereof may be included in the compositions of the disclosure. In addition, mixtures of the above alpha and beta hydroxyl acids or alpha ketoacids.

Non-limiting examples of antibacterial agents include bacitracin, phenol, benzethonium chloride, erythromycin, neomycin, tetracycline, chlortetracycline and mixtures thereof.

Non-limiting examples of pH adjusting agents includes potassium acetate, sodium carbonate, sodium hydroxide, phosphoric acid, succinic acid, sodium citrate, citric acid, boric acid, lactic acid, sodium hydrogen carbonate and mixtures thereof.

Bleaching agents include, but not limited to, hydrogen peroxide, perborate and persufate salts. EDTA and other aminocarboxylates may be used as sequestering agents. Anti-dandruff agents such as zinc pyrithione, salicylic acid, climbazole, ketoconazole, sulfur piroctone olamine, selenium sulfide and mixtures thereof may also be used as an auxiliary ingredient. Examples of suitable film forming agents useful in the compositions of the disclosure include:
a) sulfopolyester resins, such as AQ sulfopolyester resins, such as AQ29D, AQ35S, AQ38D, AQ38S, AQ48S, and AQ55S (available from Eastman Chemicals);
b) polyvinylacetate/polyvinyl alcohol polymers, such as Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) polyvinylpyrrolidones (PVP), including Luviskol K17, K30 and K90 (available from BASF), water soluble copolymers of PVP, including PVP/VA S-630 and W-735 and PVP/dimethylaminoethylmethacrylate Copolymers such as Copolymer 845 and Copolymer 937 available from ISP, as well as other PVP polymers disclosed by E. S. Barabas in the Encyclopedia of Polymer Science and Engineering, 2 Ed., Vol. 17, pp. 198-257;
e) high molecular weight silicones such as dimethicone and organic-substituted dimethicones, especially those with viscosities of greater than about 50,000 mPas;
f) high molecular weight hydrocarbon polymers with viscosities of greater than about 50,000 mPas;
g) silicone-acrylate copolymers, including VS-70 (3M), SA-70 (3M), KP-545 (Shin-Etsu)
h) organosiloxanes, including organosiloxane resins, fluid diorganopolysiloxane polymers and silicone ester waxes;
i) polyurethanes, including Polyderm series of polymers from Alzo, Corp.; and
j) hydrophobic acrylate copolymers, including the acrylate/alkylmethacrylate copolymer Lipacryl (Rohm & Haas) or its emulsified, water dispersible version Allianz OPT (ISP).

The compositions of the disclosure may comprise one or more absorbent materials. These absorbents are useful for achieving the uptake of various fluids that are commonly present on the skin, e.g., perspiration, oil, and/or sebum. Suitable absorbents include, but are not limited to, silicas, silicates, polyacrylates, cross-linked silicones, cross-linked hydrocarbons, activated carbon, starch-based materials (for example cornstarch (topical starch), talc, rice starch, oat starch, tapioca starch, potato starch, legume starches, soy starch, turnip starch), microcrystalline cellulose (for example Avicel®), aluminum starch octenyl succinate (sold by National Starch & Chemical Co. as Dry Flo® Pure, Dry Flo® XT, Dry Flo® PC, and/or Dry Flo® AF (aluminum free grade)), kaolin, calcium silicate, amorphous silicas, calcium carbonate, magnesium carbonate, or zinc carbonate, and mixtures thereof. Some specific examples of the silicates and carbonates useful in the present invention are more fully explained in Van Nostrand Reinhold's Encyclopedia of Chemistry. 4th Ed. pages 155, 169,556, and 849, (1984).

Examples of useful anti-acne actives of the disclosure include, but are not limited to, the keratolytics such as salicylic acid (o-hydroxybenzoic acid), derivatives of salicylic acid such as 5-octanoyl salicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids; and bile salts such as scymnol sulfate and its derivatives, deoxycholate, and cholate.

Antiperspirant actives may also be included in the compositions of the disclosure. Suitable antiperspirant actives include astringent metallic salts, especially the inorganic and organic salts of aluminum zirconium and zinc, as well as mixtures thereof. Particularly preferred are the aluminum containing and/or zirconium-containing materials or salts, such as aluminum halides, aluminum chlorohydrate, aluminum hydroxyhalides, zirconyl oxyhalides, zirconyl hydroxyhalides, and mixtures thereof.

Examples of anti-wrinkle and anti-skin atrophy actives useful in the disclosure include, but are not limited to, retinoic acid and its derivatives (e.g., cis and trans); retinol; retinyl esters; niacinamide, and derivatives thereof; sulfur-containing D and L amino acids and their derivatives and salts, particularly the N-acetyl derivatives, a preferred example of which is N-acetyl-L-cysteine; thiols (e.g., ethane thiol); terpene alcohols (e.g., farnesol); hydroxy acids, phytic acid, lipoic acid; lysophosphatidic acid, alpha-hydroxy acids (e.g., lactic acid and glycolic acid), beta-hydroxy acids (e.g., salicylic acid), and skin peel agents (e.g., phenol and the like).

Examples of artificial tanning actives and accelerators useful in the compositions of the disclosure include, but are not limited to, dihydroxyacetaone, tyrosine, tyrosine esters such as ethyl tyrosinate, phospho-DOPA, and mixtures thereof.

The compositions of disclosure may include astringents. Astringents are useful for shrinking pores of the skin. Suitable astringents include, but are not limited to, clove oil, fomes officinalis extract, spiraea ulmaria extract, menthol, camphor, eucalyptus oil, eugenol, menthyl lactate, witch hazel distillate, aluminum salts, tannins, ethanol, and combinations thereof.

The disclosed composition can also comprise or more hydrophilic conditioning agents. Non-limiting examples of hydrophilic conditioning agents include those selected from the group consisting of polyhydric alcohols, polypropylene glycols, polyethylene glycols, ureas, pyrolidone carboxylic acids, ethoxylated and/or propoxylated C₃-C₆ diols and triols, alpha-hydroxy C₂-C₆ carboxylic acids, ethoxylated and/or propoxylated sugars, polyacrylic acid copolymers, sugars having up to about 12 carbons atoms, sugar alcohols having up to about 12 carbon atoms, and mixtures thereof Specific examples of useful hydrophilic conditioning agents include materials such as urea; guanidine; glycolic acid and glycolate salts (e.g., ammonium and quaternary alkyl ammonium); lactic acid and lactate salts (e.g., ammonium and quaternary alkyl ammonium); sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-30, PEG-50, polypropylene glycols such as PPG-9, PPG-12, PPG-15, PPG-17, PPG-20, PPG-26, PPG-30, PPG-34; alkoxylated glucose; hyaluronic acid; cationic skin conditioning polymers (e.g., quaternary ammonium polymers such as Polyquaternium polymers); and mixtures thereof Glycerol, in particular, is a preferred hydrophilic conditioning agent in the present invention. Also useful are materials such as aloe vera in a variety of forms (e.g., aloe vera gel), chitosan and chitosan derivatives (e.g., chitosan lactate, lactamide monoethanolamine); acetamide monoethanolamine; and mixtures thereof. Also useful are propoxylated glycerols as described in propoxylated glycerols described in U.S. Pat. No.4,976,953.

The composition may comprise one or more hydrophobic conditioning agents. Preferred hydrophobic conditioning agents are selected from the group consisting of mineral oil, petrolatum, lecithin, hydrogenated lecithin, lanolin, lanolin derivatives, C₇-C₄₀ branched chain hydrocarbons, C₁-C₃₀ alcohol esters of C₁-C₃₀ carboxylic acids, C₁-C₃₀ alcohol esters of C₂-C₃₀ dicarboxylic acids, monoglycerides of C₁-C₃₀ carboxylic acids, diglycerides of C₁-C₃₀ carboxylic acids, triglycerides of C₁-C₃₀ carboxylic acids, ethylene glycol monoesters of C₁-C₃₀ carboxylic acids, ethylene glycol diesters of C₁-C₃₀ carboxylic acids, propylene glycol monoesters of C₁-C₃₀ carboxylic acids, propylene glycol diesters of C₁-C₃₀ carboxylic acids, C₁-C₃₀ carboxylic acid monoesters and polyesters of sugars, polydialkylsiloxanes, polydiarylsiloxanes, polyalkylarylsiloxanes, cyclomethicones having 3 to 9 silicone atoms, vegetable oils, hydrogenated vegetable oils, polypropylene glycol C₄-C₂₀ alkyl ethers, di C₈-C₃₀ alkyl ethers, and combinations thereof.

The compositions may comprise a light diffuser. Light diffusers are useful for improving skin appearance by minimizing the appearance of texture such as pores and fine lines. Suitable light diffusers for inclusion into the compositions of the present system include, but are not limited to silica, nylon, polyethylene, polymethyl methacrylate, polystyrene, methylsiloxane copolymer, polytetrafluoroethylene copolymer, boron nitride, silicone resin powders, silicone rubber powders, ethylene acrylate copolymers, mica, titanium dioxide, iron oxides, zinc oxide, and combinations thereof.

The compositions of the disclosure may optionally comprise one or more polymeric materials that are oil-soluble and form a gel with hydrophobic materials (e.g., oils) that are contained in the compositions. Such polymers are beneficial for structuring these materials resulting in flexible gels with improved stability and shear-resistance.

Particularly suitable are at least partially cross-linked oil-soluble polymeric materials with a softening point <160° C. Suitable materials come from the chemical groups of PE (polyethylenes), PVA (polyvinyl alcohols) and derivatives, PVP (polyvinylpyrrolidones) and derivatives, PVP/Alkene Copolymers, PVP/VA copolymers, PVM/MA (methyl vinyl ether/maleic anhydride) copolymers and their esters and ethers, particularly poly (alkyl vinyl ether-co-maleic anhydride) copolymers, ethylene/VA copolymers, acrylates/alkyl methacrylate copolymer, styrene/isoprene, styrene/ethylene/butylene, styrene/ethylene/propylene, styrene/ethylene/butylene/styrene, styrene/butadiene copolymers, benotnite clays, hectorite clays, organix waxes and silicone waxes. Suitable materials are available e.g. from Dupont (ELVAX® types), BASF (LUVISKOL® types), Shell (KRATON® polymers), ISP (PVP, GANTREZ®, GANEX® and ALLIANZ OPT® types) and Rohm & Haas (LIPACRYL®).

The compositions of the disclosure may optionally contain a hydrophilic gelling agent. The gelling agent preferably has a viscosity (1% aqueous solution, 20° C., Brookfield RVT) of at least about 4000 mPas, more preferably at least about 10,000 mPas and even more preferably at least 50,000 mPas.

Suitable hydrophilic gelling agents can generally be described as water-soluble or colloidally water-soluble polymers, and include cellulose ethers (e.g. hydroxyethyl cellulose, methyl cellulose, hydroxypropylmethyl cellulose), bentonite clays, hectorite clays, polyvinylpyrrolidone, polyvinylalcohol, polyquaternium-10, guar gum, hydroxypropyl guar gum, and xanthan gum.

Among suitable hydrophilic gelling agents are acrylic acid/ethyl acrylate copolymers and the carboxyvinyl polymers sold by the B. F. Goodrich Company under the trademark of Carbopol resins. These resins consist essentially of a colloidally water-soluble polyalkenyl polyether crosslinked polymer of acrylic acid crosslinked with from about 0.75% to about 2.00% of a crosslinking agent such as for example polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid crosslinked with about 1% of a polyallyl ether of sucrose having an average of about 5.8 allyl groups for each sucrose molecule. Also suitable for use herein are hydrophobically-modified cross-linked polymers of acrylic acid having amphipathic properties available under the Trade Name Carbopol 1382, Carbopol 1342 and Pemulen TR-1 (CTFA Designation: Acrylates/10-30 Alkyl Acrylate Crosspolymer). A combination of the polyalkenyl polyether cross-linked acrylic acid polymer and the hydrophobically modified cross-linked acrylic acid polymer is also suitable for use herein. Other suitable gelling agents suitable for use herein are oleogels such as trihydroxystearin and aluminium magnesium hydroxy stearate. The gelling agents herein are particularly valuable for providing excellent stability characteristics over both normal and elevated temperatures.

Neutralizing agents suitable for use in neutralizing acidic group containing hydrophilic gelling agents herein include sodium hydroxide, potassium hydroxide, ammonium hydroxide, monoethanolamine, diethanolamine and triethanolamine.

The composition of the disclosure may optionally include a polymer that is non-linear in nature. Suitable polymers for inclusion in the claimed compositions include, but are not limited to polysiloxanes that are crosslinked organopolysiloxane polymer gel networks. For instance, particularly well-suited crosslinked organopolysiloxane polymer gel networks are formed from polymerization of an epoxy functional organosiloxane in the presence of an acid catalyst. The organopolysiloxane polymer is a crosslinked organopolysiloxane polymer gel network selected from non-emulsifying polymer gel networks, emulsifying polymer gel networks, and combinations thereof.

The following examples are for illustrative purposes only and are not intended to limit the scope of the claims. In the following examples, Lupasol® G-35 is 50% active and Carbopol® aqua CC is 20% active.

### Determination of the Amine Number and the Acid Number

The measurement of the Acid and the Amine Value is performed through a common acid-base titration in the presence of a color indicator. The method is based on the European and American Pharmacopoeias and Standard ISO 660.

Specifically, the acid value measures the quantity of free acid functions titratable with NaOH using Phenolphthalein as an indicator (the endpoint is determined by the slight pink color that persists for at least 15 seconds), and is reported as milliequivalent of acid per grams (meq/g) of the acid substance.

Similarly, the amine value measures the quantity of amine functions titratable with HCl using Bromophenol Blue as an indicator (the endpoint is determined by the slight blue color that persists for at least 15 seconds), and is reported as milliequivalent of amine per gram (meq/g) of the polyamine.

### General Procedure for Preparing the Composition of the Disclosure

The at least one acid, at least one water-insoluble ingredient and other optional oil-based ingredients are mixed at a temperature of at least 25° C. in a container A. The at least one polyamine, water and other optional ingredients are mixed at a temperature of at least 25° C. in a container B. Next the contents of container B is slowly added to container A with high shear mixing. After all of container B is added, other optional ingredients described above are added while mixing at high shear. Mixing continues until a homogeneous mixture is obtained.

### A. General Test for the Measurement of the Water-Resistance of Disclosed Composition

The water-resistance of a surface treated with the disclosed composition can be measured using a Contact Angle Measurement System K-12 manufactured by Kruss (Germany). This instrument allows one to calculate the degree of water-resistance of a solid surface when it was pushed in and pulled out of water by measuring the angle formed by the water-solid interface. The low contact angle denotes a low water-resistance (water spreads on the surface), and the high contact angle denotes a high water-resistance (water beads on the surface).

In this test, a microscope cover glass (Fisher brand 12-542A, 18 cm×18 cm×0.16 mm) was treated with a solution of the disclosed composition (50 g of Isopropanol (IPA)+10 g of the disclosed composition) by dipping the cover glass to half of its length into the testing solution and allowing it to dry. The treated cover glass is then mounted on the Kruss instrument and the Advancing Contact Angle (Wetting Contact Angle)/Receding Contact Angle (De-wetting Contact Angle) measured using the following parameters:

Measuring Speed: 3 mm/min

Max Immersion Depth: 5 mm

Min Immersion Depth: 0 mm

Sensitivity: 0.01 g

### B. Measurements of Contact Angle for Mixtures Containing Components (a), (b), (c) and (e)

Following the General procedure described in this above, the Contact Angle of the following compositions were measured (n=5) (Table 1-1):

TABLE 1-1

| | Oleic | | Mineral | | | Advancing | Receding |
|---|---|---|---|---|---|---|---|
| | Acid | Lupasol ® | Oil | Procetyl | Amine #: | Contact Angle | Contact Angle |
| IPA (%) | (%) | G35 (%) | (%) | AWS (%) | Acid # | (°) | (°) |
| 98.08 | 1.04 | 0.38 | 0.5 | — | 1:1 | 92.6 ± 0.6 | 70.7 ± 0.6 |
| 99.87 | 0.10 | 0.016 | 0.008 | — | 1:0.2 | 81.9 ± 1.4 | 65.4 ± 1.7 |
| 88.08 | 1.04 | 0.38 | 0.5 | 10 | 1:1 | 74.0 ± 2.2 | 63.9 ± 0.4 |

The data in Table 1-1 show that when the concentration of the ingredients in the disclosed composition is lowered and the Amine number : Acid number is outside the claimed range, both the Advancing Contact Angle and the Receding Contact Angle decrease to below 66 degrees. A decrease in the contact angle can also be seen in a case where additional ingredients such as nonionic surfactant (Procetyl AWS) are added to the claimed composition. These results demonstrate that not all compositions necessarily have the disclosed contact angle of 66 degrees.

Table 1-2 lists the contact angles on an untreated and a disclosed composition treated glass surface (n=10):

TABLE 1-2

| Tested Disclosed Compositions | | | | | | | |
|---|---|---|---|---|---|---|---|
| | | Ratio of amine | | | | | |
| Isostearic | Lupasol ® | number to | | Water | IPA | Contact Angle (°) | |
| Acid (%) | G35 (%) | acid number | Min. Oil (%) | (%) | (%) | Advancing | Receding |
| | | | | | 100 | 9.8 ± 2.9 | 6.7 ± 1.6 |
| 0.3 | 0.13 | 1:0.84 | 0.1 | 16.47 | 83.0 | 69.1 ± 1.0 | 66.4 ± 0.3 |
| 1.5 | 0.13 | 1:4.19 | 0.1 | 15.27 | 83.0 | 91.5 ± 1.1 | 73.4 ± 1.8 |
| 5.0 | 1.0 | 1:1.81 | 2.0 | 9.0 | 83.0 | 94.4 ± 1.0 | 73.5 ± 0.6 |

The data demonstrate a significant increase in water-resistance of the glass surface upon treating with the disclosed composition as evidenced by the increase in both the Advancing Contact Angle and the Receding Contact Angle.

### 2. Disclosed Composition in Skin Treatment Formulations

### Example 2.1

A test sunscreen creme and a control sunscreen creme were made as in the formulas below:

| Test formula | | Control formula | |
|---|---|---|---|
| DI Water | q.s. | DI Water | q.s. |
| Glycerin | 3.00% | Glycerin | 3.00% |
| Xanthan Gum | 0.20% | Xanthan Gum | 0.20% |
| Cosmedia SP | 0.25% | Cosmedia SP | 0.25% |
| Mexoryl SX | 10.00% | Mexoryl SX | 10.00% |
| PEI | 5.03% | TEA | 7.13% |
| Isostearic Acid | 10.00% | Isostearic Acid | 10.00% |
| Isodecyl Neopentanoate | 10.00% | Isodecyl Neopentanoate | 10.00% |

The procedure for making the test sunscreen formula is as follows: In a main tank, add Isostearic Acid and Isodecyl Neopentanoate and mix at RT. In a side tank, add DI Water. Add slurry of Xanthan Gum, Cosmedia SP, and Glycerin into the water and mix at high speed to thicken. When thickened, add Mexoryl SX and mix at moderate speed. Next, add PEI and mix well. Add the mixture in side tank slowly into the main tank with high speed mixing and side sweeping.

The procedure for making the control sunscreen formula is as follows: In a main tank, add Isostearic Acid and Isodecyl Neopentanoate and mix at RT. In a side tank, add DI Water. Add slurry of Glycerin, Xanthan Gum, and Cosmedia SP into the water and mix at high speed to thicken. When thickened, add Mexoryl SX and mix at moderate speed. Add the mixture in side tank slowly into the main tank with high speed mixing. Lastly, add TEA and mix with side sweep.

The sunscreen formulas were evaluated for water resistancy using the following method: First, the substrate of two 2 cm×2 cm squares of white Bioskin plates (Beaulax co., LTD, Japan) was prepared. An amount of 0.02 g of the control product was spread evenly on one plate, and 0.02 g of the test product was spread evenly on the other plate. Both plates were dried for 5 minutes at ambient conditions, then each were immersed into 100 ml of DI water in a jar. The jars containing the treated plates were shaken at speed 6 for up to 2 hours, and the UV absorbance of Mexoryl SX (344 nm) in the water solutions was measured over the course of 2 hours. The amount of Mexoryl SX that leached out was calculated, and then the % leached out was calculated.

The table below shows the % of Mexoryl SX that leached out over the course of 2 hours:

| | 5 min | 15 min | 30 min | 60 min | 90 min | 120 min |
|---|---|---|---|---|---|---|
| Control | 65% | 85% | 95% | 100% | 100% | 100% |
| Test | 0% | 6% | 6% | 6% | 8% | 10% |

As a result, the % of Mexoryl SX that leached out into water for the control formula is 100% and for the test formula is 10% after 2 hours of immersion with shaking in water. The results indicate that substantial amount of sunscreen in the control leached out into the water compared to the test. Therefore the disclosed composition improves the water resistancy of the sunscreen.

### Example 2.2

A test anti-perspirant creme and a control anti-perspirant creme were made as in the formulas below:

| Test formula | | Control formula | |
|---|---|---|---|
| DI Water | q.s. | DI Water | q.s. |
| REACH AZP-908 | 16.00% | REACH AZP-908 | 16.00% |
| Superultrafine* | | Superultrafine* | |
| PEI | 3.63% | TEA | 5.15% |
| Isostearic Acid | 20.00% | Isostearic Acid | 20.00% |
| Isodecyl Neopentanoate | 10.00% | Isodecyl Neopentanoate | 10.00% |

*INCI: Aluminum Zirconium Tetrachlorohydrex Gly (15% Aluminum)

The procedure for making the control anti-perspirant formula above is as follows: In a main tank, add Isostearic Acid and Isodecyl Neopentanoate and mix at RT. In a side tank, add DI Water and Aluminum Zirconium Tetrahydrex Gly, the active ingredient of the anti-perspirant, and mix at RT. Add into main tank slowly with high speed mixing. Lastly, add TEA and mix at moderate speed.

The process for making the Test formula is as follows: In a main tank, add Isostearic Acid and Isodecyl Neopentanoate and mix at RT. In a side tank, add half the amount of the DI Water and PEI and mix at RT. Add into the main tank slowly and mix with high speed mixing. In another side tank, add the remainder of water and Aluminum Zirconium Tetrahydrex Gly and mix at RT. Add into main tank slowly and mix at moderate speed.

The anti-perspirant creme formulas were evaluated for water resistancy using the following method: First, the substrate of two 2 cm×2 cm squares of white Bioskin plates (Beaulax co., LTD, Japan) was prepared. An amount of 0.02 g of the control product was spread evenly on one plate, and 0.02 g of the test product was spread evenly on the other plate. Both plates were dried for 5 minutes at ambient conditions, then each were immersed into 100 ml of DI water in a jar. The jars containing the treated plates were shaken at speed 6 for 2 hours. The water was tested for Aluminum content using the Aluminum reagent kit (Eriochrome Cyanine R Method) from Hach, in which the absorbance at 535 nm represents the amount of Aluminum present in the water solution.

As a result, the % of Aluminum that leached out into water for the control formula is 70% and for the test formula is none. Therefore the disclosed composition improves the water resistancy of the anti-perspirant.

The foregoing description illustrates and describes the present disclosure. Additionally, the disclosure shows and describes only the preferred embodiments of the disclosure, but, as mentioned above, it is to be understood that it is capable of changes or modifications within the scope of the concept as expressed herein, commensurate with the above teachings and/or skill or knowledge of the relevant art. The embodiments described hereinabove are further intended to explain best modes known of practicing the invention and to enable others skilled in the art to utilize the disclosure in such, or other, embodiments and with the various modification required by the particular applications or uses disclosed herein. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

What is claimed:

1. A method for treating keratinous substrates comprising applying a composition to a keratinous substrate wherein the composition comprises
(a) at least one polyamine,
(b) at least one acid,
(c) at least one water-insoluble ingredient and
(d) solvent, and
(e) at least one auxiliary ingredient
wherein the molar ratio of the amine groups in the at least one polyamine to the acid groups in the at least one acid is from about 1:0.5 to about 1:30 and wherein a mixture of components (a), (b), (c) and (d) form a mixture that has a contact angle of at least about 66 degrees on glass.

2. The method as claimed in claim 1, wherein the at least one polyamine (a) is selected from the group consisting of a polyethyleneimine, a polyvinylamine, an aminated polysaccharide, an amine substituted polyalkylene glycol, an amine substituted polyacarylate crosspolymer, an amine substituted polyacrylate, an amine substituted polymethacrylate, a protein, an amine substituted polyester, a polyamino acid, an amodimethicone, a polyalkylamine, diethylene triamine, triethylenetetramine, spermidine, spermine, aminosilicone and mixtures thereof.

3. The method as claimed in claim 1, wherein the at least one polyamine (a) is selected from the group consisting of a polyethyleneimine, a polyvinylamine, chitosan, polylysine, polyacrylate-1-cross-polymer and mixtures thereof.

4. The method as claimed in claim 1, wherein the at least one acid (b) is selected from the group consisting of a fatty carboxylic acid, a fatty ether carboxylic acid, a fatty ether phosphoric acid, a fatty phosphoric acid and mixtures thereof

5. The method as claimed in claim 1, wherein the at least one acid (b) is a monoacid or a polyacid.

6. The method as claimed in claim 1, wherein the at least one acid (b) is selected from the group consisting of capric acid, caprylic acid, isosteric acid, oleic acid, stearic acid, lauric acid, linoleic acid, linolenic acid, laureth-5 carboxylic acid, laureth-11 carboxylic acid, cetyl phosphosphate, stearyl phosphate, oleth-3 phosphate, oleth-10 phosphate and mixtures thereof

7. The method as claimed in claim 1, wherein the at least one water-insoluble ingredient (c) is selected from the group consisting of an oil, a fatty ester, a hydrocarbon oil, a silicone, a wax, a fatty acid, a fatty alcohol and mixtures thereof.

8. The method as claimed in claim 1, wherein the at least one water-insoluble ingredient (c) is selected from the group consisting of olive oil, avocado oil, coconut oil, mineral oil, isopropyl palmitate, capric/caprylic triglyceride, isododecane, polyisobutene, dimethicone, phenyltrimethicone, beeswax and mixtures therefore.

9. The method as claimed in claim 1, wherein the solvent (d) is at least one selected from the group consisting of water, an alcohol, a nonaqueous polar solvent and a nonaqueous nonpolar solvent.

10. The method as claimed in claim 1, wherein the at least one auxiliary ingredient (e) is selected from the group consisting of an amino acid, a protein, a cationic conditioner, a cationic polymer, a anionic surfactant, a nonionic surfactant, an amphoteric surfactant, a zwitterionic surfactant, a viscosity modifier, an organosiloxane polymer, a wax, a silicone resin, a pigment, a powder, a preservative, an antioxidant, a vitamin, an alpha hydroxy acid, a beta hydroxy acid, an alpha ketoacid, an antibacterial agent, a sunscreen, a preservative, a pH adjusting agent, a bleaching agent, a perfume, a sequestering agent, an anti-dandruff agent, a film forming agent, an absorbant, an anti-acne active, an antiperspirant active, an anti-wrinkle and antiskin atrophy active, an artificial tanning and accelerator, an astringent, a hydrophilic conditioning agent, a hydrophobic conditioning agent, an oil-soluble gelling agent, a hydrophilic gelling agent, a crosslinked silicone polymer and mixtures thereof.

11. The method as claimed in claim 1, wherein the at least one polyamine (a) is present in a positive amount up to about 30% by weight, based on the weight of the composition.

12. The method as claimed in claim 1, wherein the at least one acid (b) is present in a positive amount up to about 50% by weight, based on the weight of the composition.

13. The method as claimed in claim 1, wherein the at least one water-insoluble ingredient (c) is present in a positive amount up to about 50% by weight, based on the weight of the composition.

14. The method as claimed in claim 1, wherein solvent (d) is present in an amount of from about 10% to about 90% by weight, based on the weight of the composition.

15. The method as claimed in claim 1, wherein the at least one auxiliary ingredient (e) is present in a positive amount up to about 50%, based on the weight of the composition.

16. A method of protecting skin from UV light damage comprising utilizing the method as claimed in claim 1.

17. A method of protecting skin from water, environmental damage, and chemical damage comprising utilizing the method as claimed in claim 1.

18. A method of reducing the appearance of wrinkles in skin comprising utilizing the method as claimed in claim 1.

19. A method of prolonging the efficacy of an active ingredient on a keratinous substrate comprising utilizing the method as claimed in claim 1.
